(19) United States
(12) Patent Application Publication
(10) Pub. No.: US 2012/0108650 A1
(43) Pub. Date: May 3, 2012

(54) MICRO RNA MARKERS AND METHODS RELATED THERETO

(75) Inventors: Peng Jin, Marietta, GA (US); Allan I. Levey, Atlanta, GA (US); Keith Szulwach, Cumming, GA (US)

(73) Assignee: EMORY UNIVERSITY, Atlanta, GA (US)

(21) Appl. No.: 13/283,953

(22) Filed: Oct. 28, 2011

Related U.S. Application Data
(60) Provisional application No. 61/408,121, filed on Oct. 29, 2010.

(51) Int. Cl. A61K 31/7088; C40B 30/04; A61P 25/28; C12Q 1/68

(52) U.S. Cl. 514/44 A; 435/6.11; 506/9

(57) ABSTRACT

The present invention provides methods of diagnosis of Alzheimer's disease including assessing the levels of certain microRNAs in a subject and comparing these to levels in subjects not exhibiting the disease. The identified measurements provide input for improved diagnoses of Alzheimer's disease as compared to certain other forms of dementias, which allows more effective treatment regimens.

### CROSS-REFERENCE TO RELATED APPLICATIONS

This application claims priority to U.S. Provisional Application No. 61/408,121, filed Oct. 29, 2010, the content of which is incorporated herein in its entirety.

This invention was made with government support under Grant No. P50 AG025688 awarded by the National Institutes of Health. The government has certain rights in the invention.

### BACKGROUND

Mature microRNAs (miRNAs) are short (20-24 nt) non-coding RNAs that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce an approximately 70-nt stem-loop precursor miRNA (pre-miRNA), which is further cleaved by the cytoplasmic Dicer ribonuclease to generate the mature miRNA and antisense miRNA star (miRNA*) products. The mature miRNA is incorporated into a RNA-induced silencing complex (RISC), which recognizes target mRNAs through imperfect base pairing with the miRNA and most commonly results in translational inhibition or destabilization of the target mRNA. MiR-650 is reported to be involved in lymphatic and distant metastasis in human gastric cancer. See Zhang et al., Biochem Biophys Res Commun., 2010, 395(2):275-80. Certain microRNAs have been implicated in Alzheimer's Diseases (AD). See Wany et al., J. Neurosci., 2008, 28(5):1213-23 and Hebert et al., Proc Natl Acad Sci USA., 2008, 105(17):6415-20.

Primary degenerative dementia of the Alzheimer's type becomes worse as it progresses, and eventually leads to death. There is no cure for the disease. Alzheimer's Diseases (AD) is predicted to affect 1 in 85 people globally by 2050. Alzheimer's disease results in the loss of neurons and synapses in the cerebral cortex and certain subcortical regions. This loss results in gross atrophy of the affected regions, including degeneration in the temporal lobe and parietal lobe and parts of the frontal cortex. Both amyloid plaques and neurofibrillary tangles are clearly visible by microscopy in brains of those afflicted by AD.

Alzheimer's disease is believed to be a protein misfolding disease (proteopathy), caused by accumulation of abnormally folded A-beta and tau proteins in the brain. Plaques are made up of beta-amyloid (A-beta or Aβ). Beta-amyloid is a fragment from a larger protein called amyloid precursor protein (APP), a transmembrane protein that penetrates through the membrane of neurons. APP is important to neuron growth, survival and post-injury repair. In Alzheimer's disease, APP is divided into smaller fragments. One of these fragments gives rise to fibrils of beta-amyloid, which form clumps that deposit outside neurons in dense formations known as senile plaques. Dominant mutation in AD have been identified in apolipoprotein E (APOE) and APP, presenilin 1 (PSEN1) and presenilin 2 (PSEN2).

The cholinergic hypothesis, proposes that AD is caused by reduced synthesis of the neurotransmitter acetylcholine. The cholinergic hypothesis has not maintained widespread support because medications intended to treat acetylcholine deficiency have not been effective. The amyloid hypothesis postulated that amyloid beta (Aβ) deposits are the fundamental cause of the disease. Support for this postulate comes from the location of the gene for the amyloid beta precursor protein (APP) on chromosome 21, together with the fact that people with trisomy 21 (Down Syndrome) who have an extra gene copy almost universally exhibit AD by 40 years of age. APOE4, a genetic risk factor for AD, leads to excess amyloid buildup in the brain before AD symptoms arise. Thus, Aβ deposition typically precedes clinical AD. Further evidence comes from the finding that transgenic mice that express a mutant form of the human APP gene develop fibrillar amyloid plaques and Alzheimer's-like brain pathology with spatial learning deficits.

Non-plaque Aβ oligomers may be the primary pathogenic form of Aft Amyloid-derived diffusible ligands (ADDLs), bind to a surface receptor on neurons and change the structure of the synapse, thereby disrupting neuronal communication. N-APP, a fragment of APP from the peptide's N-terminus, is adjacent to beta-amyloid and is cleaved from APP by one of the same enzymes. N-APP triggers a self-destruct pathway by binding to a neuronal receptor called death receptor 6 (DR6, also known as TNFRSF21). DR6 is highly expressed in the human brain regions most affected by Alzheimer's, so it is possible that the N-APP/DR6 pathway might be the cause damage.

Alzheimer's disease is usually diagnosed clinically from the patient history, collateral history from relatives, and clinical observations, based on the presence of characteristic neurological and neuropsychological features and the absence of alternative conditions. Advanced medical imaging with computed tomography (CT) or magnetic resonance imaging (MRI), and with single photon emission computed tomography (SPECT) or positron emission tomography (PET) can be used to help exclude other cerebral pathology or subtypes of dementia. However, there is a need to identify improved methods of diagnosis for this disorder.

### SUMMARY

In certain embodiments, the disclosure relates to methods of screening a subject for Alzheimer's Disease comprising: a) measuring the quantity of microRNA in a sample and b) comparing the measured amount of microRNA to a predetermined amount of microRNA indicative of Alzheimer's disease. In certain embodiments the method further comprises the step of determining whether the sample is indicative of Alzheimer's disease. In certain embodiments the method further comprises the step of recording whether the sample is indicative of Alzheimer's disease.

In certain embodiments, the microRNA is one or more selected from the group miR-515-3p, miR-21, miR-576, miR-490, miR-187, miR-449, miR-646, miR-409-5p, miR-518e, miR-517c, miR-320, miR-564, miR-191, miR-142-5p, miR-501, miR-519e, miR-489, miR-124a, miR-218, and miR-650. In certain embodiments, two or more microRNA are selected or three or more microRNA are selected.

In certain embodiments, the sample is collected from a subject suspected of or at risk of having Alzheimer's disease. In certain embodiments, the sample is a blood sample obtained from pulmonary or systemic circulation. In other embodiments, the subject is provided a means for measuring the quantity of an microRNA through external means, such as through imaging using MRI, PET, or similar measurement. Measuring the quantity of microRNA and microRNA expression patterns can be done by a variety of methods including amplifying and/or sequencing one, two, three or more of nucleic acids of SEQ ID NO: 1-20 or unique portions, e.g., greater than 30, 20, 10 nucleotide segment thereof. In certain embodiments, the methods of quantifying the microRNA are performed and recorded on a computer. Determinations of related quantities may be performed by a computer one or more algorithm and stored on a computer database. In certain instances, the predetermined amount of microRNA is an amount determined from the same subject at a different time period. In another instance, the predetermined amount is derived from a control subject that is not exhibiting dementia. In certain instances, the subject is administered a therapeutic agent for treatment or prevention of alzheimer's disease prior to measurement of the microRNA.

In certain embodiments, the methods contemplate reporting the test results to the subject from which the sample was taken or doctor or other medical professional providing care or advice to the subject verbally or written or electronic documents.

In certain embodiments, the disclosure relates to a method of screening a subject for Alzheimer's disease (AD), comprising: detecting an amount of one or more markers associated with AD in a biological sample from said subject, wherein said one or more markers is selected from the group consisting of miR-125a, miR-187, miR-196a, miR-486, miR-490, miR-572 and miR-650, any combination thereof, repeating such detection at a later time period, wherein detection of an increase in the amount of said one or more markers identifies the subject as having AD.

In certain embodiments, the disclosure relates to a method of screening a subject for AD, comprising: detecting an increase in an amount of one or more markers associated with AD in a biological sample from said subject, wherein said one or more markers is selected from the group consisting of miR-125a, miR-187, miR-196a, miR-486, miR-490, miR-572 and miR-650, whereby detection of an increase in said one or both markers identifies the subject as having an increased risk of developing AD.

In certain embodiments, the disclosure relates to a method of diagnosing AD in a subject, comprising: detecting an increase in an amount of one or more markers associated with AD in a biological sample from said subject, wherein said one or more markers is selected from the group consisting of miR-125a, miR-187, miR-196a, miR-486, miR-490, miR-572 and miR-650, whereby detection of an increase in said one or more markers diagnoses the subject as having AD.

In certain embodiments, the disclosure relates to a method of identifying a subject as having AD, comprising: detecting an increase in an amount of one or more miRNAs in said subject, wherein said miRNA is selected from the group consisting of miR-125a, miR-187, miR-196a, miR-486, miR-490, miR-572 and miR-650, individually or in combination thereof, whereby detection of an increase in said one or more miRNAs identifies the subject as having AD.

In certain embodiments, the disclosure relates to a method of identifying a subject as having an increased risk of developing AD, comprising: detecting an increase in an amount of one or more miRNAs in said subject, wherein said miRNA is selected from the group consisting of miR-125a, miR-187, miR-196a, miR-486, miR-490, miR-572 and miR-650, whereby detection of an increase in said one or more miRNAs identifies the subject as having an increased risk of developing AD.

In certain embodiments, the disclosure relates to a method of diagnosing or prognosticating AD in a subject, comprising: i) determining the level of at least one miR gene product in a sample containing human tissue, blood, serum or cerebrospinal fluid from the subject; and ii) comparing the level of at least one miR gene product in the sample to a control, wherein an increase in the level of at least one miR gene product in the sample from the subject, relative to that of the control, is diagnostic or prognostic of AD.

In certain embodiments, the control is selected from the group consisting of: i) a reference standard, ii) the level of at least one miR gene product from a subject that does not have AD, and iii) the level of at least one miR gene product from a sample of the subject that does not exhibit AD but may or may not display signs of dementia such as frontotemporal dementia.

In certain embodiments, the subject is a human.

In certain embodiments, the alteration is an increase in the level of miR gene products, namely miR-490, miR-187, miR-196a, miR-486 and miR-125a.

In certain embodiments, the alteration is a decrease in the level of miR gene products, namely miR-30d, miR-103, miR-135a, miR-365, miR-130b, miR-98, miR-195, miR-30e-5p, let-7e, miR-135b and let-7f.

In certain embodiments, the method comprises assaying a sample of miRNA by sequencing the miRNA, hybridizing the miRNA with a complementary nucleic acid and detecting the hybridization, e.g., dye that intercalates into a double helix, dye conjugated to the complementary nucleic acid, disrupting FRET.

In certain embodiments, the disclosure relates to a method of modulating gene expression in a cell comprising administering to the cell an amount of an isolated nucleic acid comprising a miR-650 nucleic acid sequence in an amount sufficient to modulate the expression of APOE, beta-synuclein (SNCB), CDK5 and PSEN1.

In certain embodiments, the cell is in a subject having, suspected of having, or at risk of developing a nervous system disorder.

In certain embodiments, the nervous system disorder is Alzheimer-type dementia selected from the group consisting of Alzheimer's disease, vascular dementia, fronto-temporal dementia, Pick's disease and Lewy's bodies disease.

In certain embodiments, the cell is a brain, a glial, a neuronal, or a blood cell.

In certain embodiments, the disclosure relates to a composition comprising: an RNAi-inducing entity, wherein the RNAi-inducing entity is targeted to the CdkS transcript; and a delivery agent selected from the group consisting of: cationic polymers, modified cationic polymers, peptide molecular transporters, liposomes, non-cationic polymers, modified non-cationic polymers, nanoparticles or viral vectors.

In certain embodiments, the disclosure relates to a recombinant vector comprising a microRNA discloses herein wherein the vector is a retrovirus, adenovirus, lentivirus, baculovirus, or adeno-associated-virus (AAV).

### BRIEF DESCRIPTION OF THE FIGURES

FIG. 1 shows data suggesting altered expression of selective miRNAs in AD brains when compared to those with frontotemporal dementia (FTD).

FIG. 2 shows data suggesting altered processing of miR-650 in AD brains.

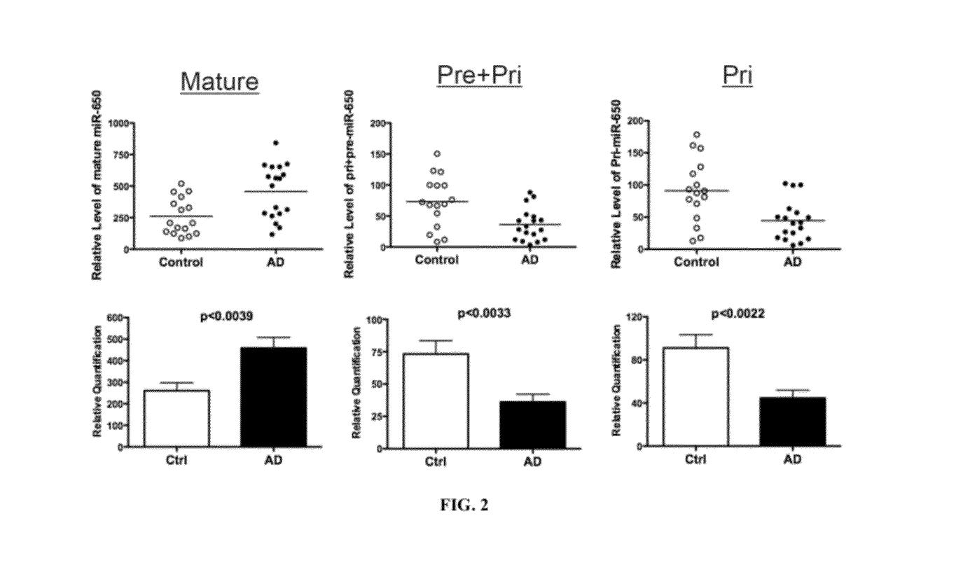

FIG. 3 shows data suggesting miR-650 posttranscriptionally regulates multiple genes implicated in AD pathogenesis.

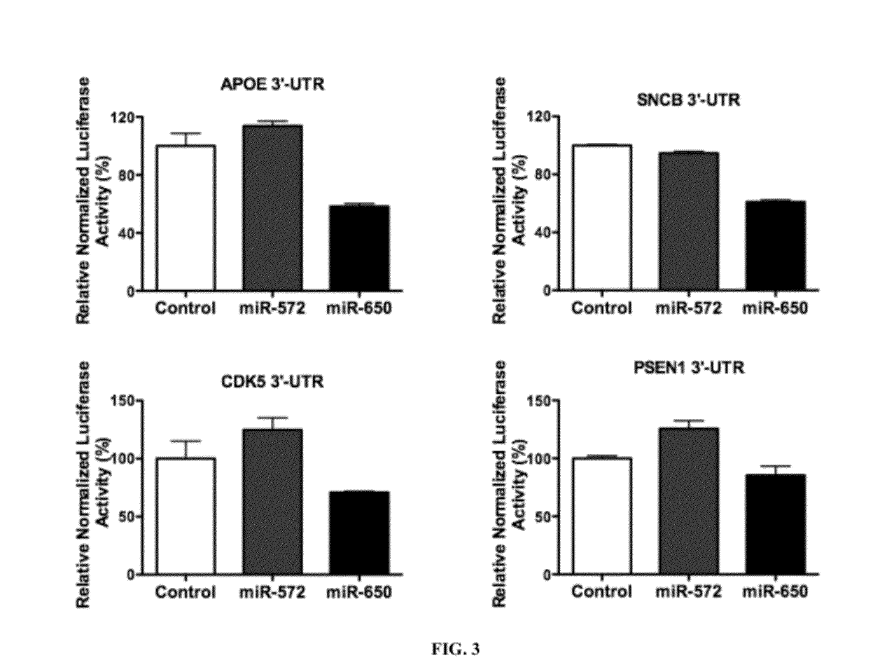

FIG. 4A illustrates a study done to evaluate the effects of miR-650 overexpression using a recombinant vector.

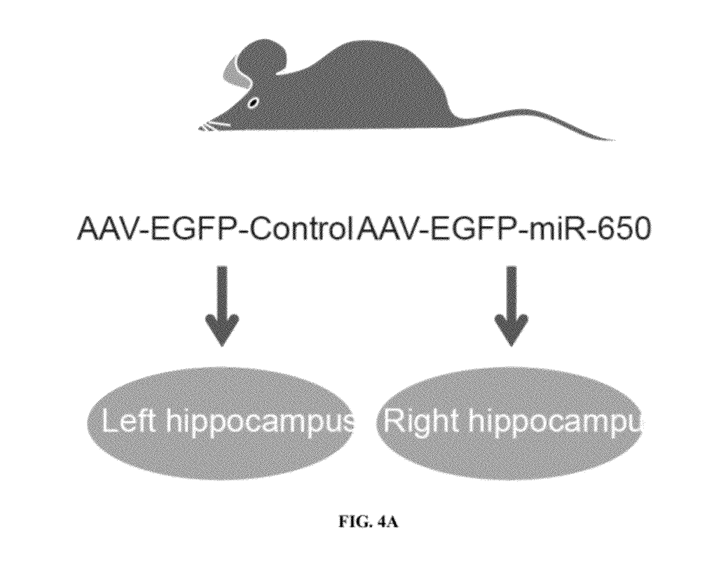

FIG. 4B shows data suggesting overexpression of miR-650 in hippocampus inhibits the expression of CDK5.

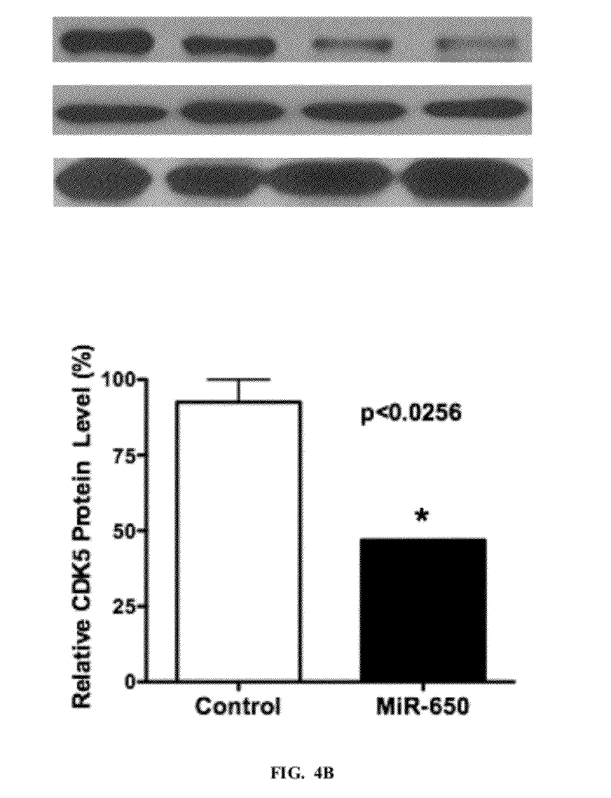

FIG. 5 shows data suggesting miR-650 is of higher relative quantity in the blood samples of AD patients.

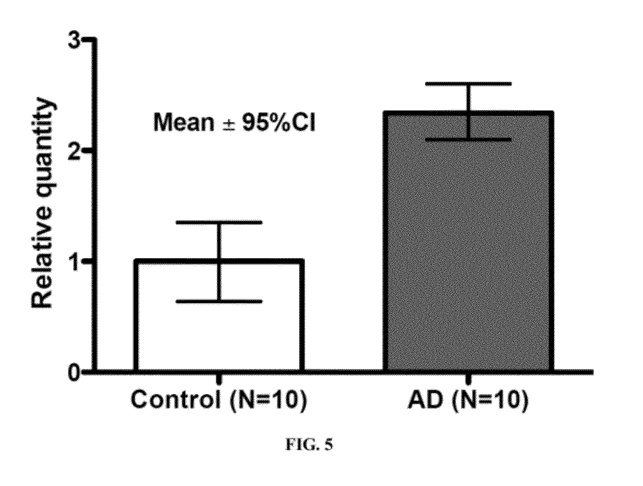

FIG. 6 MiR-650 regulates the expression of APOE post-transcriptionally. A. MiR-650 target site within the 3′-UTR of APOE mRNA. B. MiR-650, but not miR-572 suppressed the expression of the reporter gene with the APOE 3′-UTR. C. The suppression is miR-650-dependent.

FIG. 7 shows a table of data related to micro-RNA expression for control and AD patients.

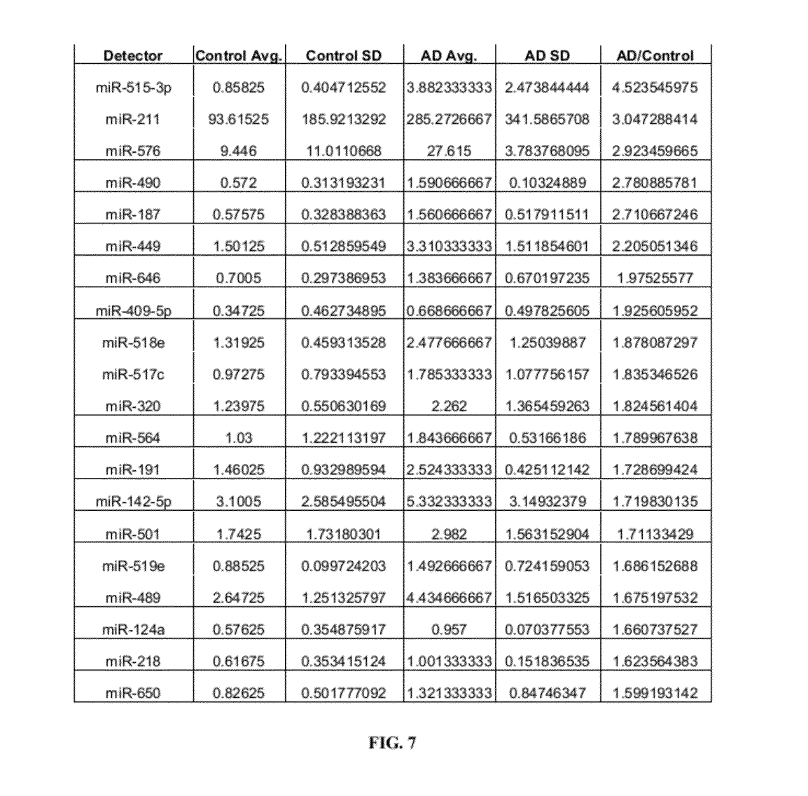

FIG. 8 shows experimental data. A. The Protein level of β-synuclein is reduced in AD brain tissues. B. Relative expression levels of miR-650 and β-synuclein protein levels in control and AD brain tissues.

FIG. 9 illustrates interactions of mi-650. A. PSEN1 and CDK5 mRNAs are predicted to be regulated by miR-650 as well. B. Additional miRNAs with altered expression in AD brain tissues and their potential mRNA targets that have been implicated in AD pathogenesis.

FIG. 10 shows data suggesting overexpression of miR-650 in APP-PSEN1 double transgene mice also decreases CDK5 expression.

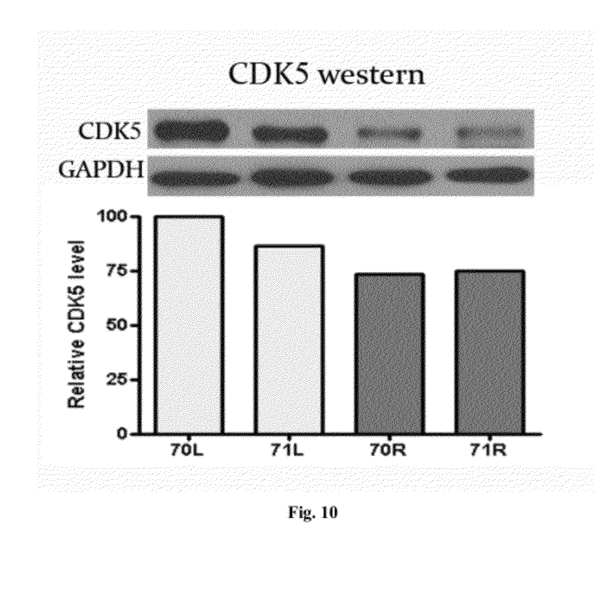

### DETAILED DESCRIPTION

### Methods of Use

In certain embodiments, the disclosure relates to methods of screening a subject for Alzheimer's disease comprising a) measuring the quantity of microRNA in a sample providing a measured amount of microRNA and b) comparing the measured amount of microRNA to a predetermined amount of microRNA indicative of Alzheimer's Disease. In certain embodiments the method further comprises the step of determining whether the sample is indicative of Alzheimer's disease. In certain embodiments the method further comprises the step of recording whether the sample is indicative of Alzheimer's disease. In other embodiments, the method further includes, if the measured amount of microRNA is elevated over the predetermined amount, then administering an Alzheimer's disease treatment to the subject. The method may also include if the measured amount is not elevated, testing the subject for cardiovascular disease, Frontotemporal dementia (FTD), or depression.

In certain embodiments, the measured quantity of microRNA is at least one times, or least two times, or at least three times, or at least four times, or at least five times the level of the measured control.

In certain embodiments, the microRNA is one or more selected from the group miR-515-3p, miR-21, miR-576, miR-490, miR-187, miR-449, miR-646, miR-409-5p, miR-518e, miR-517c, miR-320, miR-564, miR-191, miR-142-5p, miR-501, miR-519e, miR-489, miR-124a, miR-218, and miR-650. In certain embodiments, two or more microRNA are selected or three or more microRNA are selected. In certain embodiments, the sample is collected from a subject suspected of or at risk of having Alzheimer's disease. In certain embodiments, the sample is a blood sample obtained from pulmonary or systemic circulation. Measuring the quantity of microRNA and microRNA expression patterns can be done by a variety of methods including amplifying and/or sequencing one, two, three or more of nucleic acids of SEQ ID NO: 1-20 or unique portions, e.g., greater than 30, 20, 10 nucleotide segment thereof.

In certain embodiments, a subject is administered an agent that binds a microRNA as described herein to the subject. The agent can be a complementary nucleic acid to at least 50%, or at least 60%, or at least 70%, or at least 80%, or at least 90%, or at least 95% or more of any one of the microRNA's of SEQ ID NO: 1-20. In certain embodiments, the administration of such an agent is directly into a brain region to avoid systemic circulation. In certain embodiments, such a microRNA is encapsulated to avoid degradation. In certain embodiments, the administration of the agent is to diagnose Alzheimer's disease. In certain other instances, the administration is to reduce expression of such microRNA.

### MicroRNA

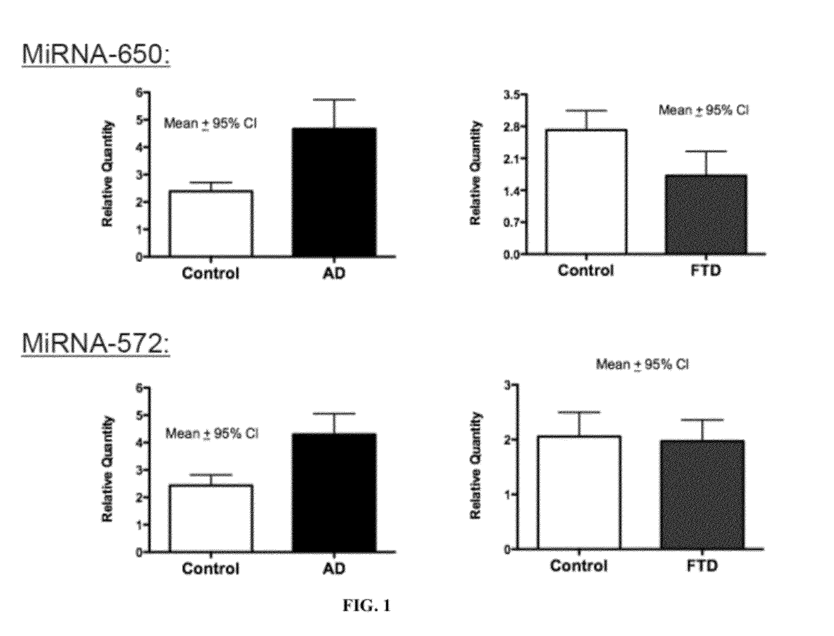

MicroRNA expression profiles have been investigated in the brain tissues from AD patients. It has been discovered that certain micro RNA, for example miRNA-650, are altered in patients with AD (See FIG. 1).

In certain embodiments, the microRNA is one or more selected from the group miR-515-3p, miR-21, miR-576, miR-490, miR-187, miR-449, miR-646, miR-409-5p, miR-518e, miR-517c, miR-320, miR-564, miR-191, miR-142-5p, miR-501, miR-519e, miR-489, miR-124a, miR-218, and miR-650. In certain embodiments, two or more microRNA are selected or three or more microRNA are selected. In certain embodiments, the sample is collected from a subject suspected of or at risk of having Alzheimer's disease. In certain embodiments, the sample is a blood sample obtained from pulmonary or systemic circulation. Measuring the quantity of microRNA and microRNA expression patterns can be done by a variety of methods including amplifying and/or sequencing one, two, three or more of nucleic acids of SEQ ID NO: 1-20 or unique portions, e.g., greater than 30, 20, 10 nucleotide segment thereof

(SEQ ID NO: 1)
Human pre-miRNA-21-650 is CAGTGCTGGG GTCTCAGGAG
GCAGCGCTCT CAGGACGTCA CCACCATGGC CTGGGCTCTG
CTCCTCCTCA CCCTCCTCAC TCAGGGCACA GGTGAT.
(SEQ ID NO: 2)
Human pre-miRNA-515 is TCTCATGCAG TCATTCTCCA
AAAGAAAGCA CTTTCTGTTG TCTGAAAGCA GAGTGCCTTC
TTTTGGAGCG TTACTGTTTG AGA.
(SEQ ID NO: 3)
Human pre-miRNA-21 is TGTCGGGTAG CTTATCAGAC
TGATGTTGAC TGTTGAATCT CATGGCAACA CCAGTCGATG
GGCTGTCTGA CA.
(SEQ ID NO: 4)
Human pre-miRNA-576 is TACAATCCAA CGAGGATTCT
AATTTCTCCA CGTCTTTGGT AATAAGGTTT GGCAAAGATG
TGGAAAAATT GGAATCCTCA TTCGATTGGT TATAACCA.
(SEQ ID NO: 5)
Human pre-miRNA-490 is TGGAGGCCTT GCTGGTTTGG
AAAGTTCATT GTTCGACACC ATGGATCTCC AGGTGGGTCA
AGTTTAGAGA TGCACCAACC TGGAGGACTC CATGCTGTTG
AGCTGTTCAC AAGCAGCGGA CACTTCCA.
(SEQ ID NO: 6)
Human pre-miRNA-187 is GGTCGGGCTC ACCATGACAC
AGTGTGAGAC CTCGGGCTAC AACACAGGAC CCGGGCGCTG
CTCTGACCCC TCGTGTCTTG TGTTGCAGCC GGAGGGACGC
AGGTCCGCA.
(SEQ ID NO: 7)
Human pre-miR-449a is CTGTGTGTGA TGAGCTGGCA
GTGTATTGTT AGCTGGTTGA ATATGTGAAT GGCATCGGCT
AACATGCAAC TGCTGTCTTA TTGCATATAC A.
(SEQ ID NO: 8)
Human pre-miR-646 is GATCAGGAGT CTGCCAGTGG
AGTCAGCACA CCTGCTTTTC ACCTGTGATC CCAGGAGAGG
AAGCAGCTGC CTCTGAGGCC TCAGGCTCAG TGGC.
(SEQ ID NO: 9)
Human pre-miR-409 is TGGTACTCGG GGAGAGGTTA
CCCGAGCAAC TTTGCATCTG GACGACGAAT GTTGCTCGGT
GAACCCCTTT TCGGTATCA.
(SEQ ID NO: 10)
Human pre-miR-518e is TCTCAGGCTG TGACCCTCTA
GAGGGAAGCG CTTTCTGTTG GCTAAAAGAA AAGAAAGCGC
TTCCCTTCAG AGTGTTAACG CTTTGAGA.
(SEQ ID NO: 11)
Human pre-miR-517c is GAAGATCTCA GGCAGTGACC
CTCTAGATGG AAGCACTGTC TGTTGTCTAA GAAAAGATCG
TGCATCCTTT TAGAGTGTTA CTGTTTGAGA AAATC.
(SEQ ID NO: 12)
Mouse pre-miR-320 is GCCTCGCCGC CCTCCGCCTT
CTCTTCCCGG TTCTTCCCGG AGTCGGGAAA AGCTGGGTTG
AGAGGGCGAA AAAGGATGTG GG.
(SEQ ID NO: 13)
Human pre-miR-564 is CGGGCAGCGG GTGCCAGGCA
CGGTGTCAGC AGGCAACATG GCCGAGAGGC CGGGGCCTCC
GGGCGGCGCC GTGTCCGCGA CCGCGTACCC TGAC.
(SEQ ID NO: 14)
Human pre-miR-191 is CGGCTGGACA GCGGGCAACG
GAATCCCAAA AGCAGCTGTT GTCTCCAGAG CATTCCAGCT
GCGCTTGGAT TTCGTCCCCT GCTCTCCTGC CT.
(SEQ ID NO: 15)
Human pre-miR-142 is GACAGTGCAG TCACCCATAA
AGTAGAAAGC ACTACTAACA GCACTGGAGG GTGTAGTGTT
TCCTACTTTA TGGATGAGTG TACTGTG.
(SEQ ID NO: 16)
Human pre-miR-501 is GCTCTTCCTC TCTAATCCTT
TGTCCCTGGG TGAGAGTGCT TTCTGAATGC AATGCACCCG
GGCAAGGATT CTGAGAGGGT GAGC.
(SEQ ID NO: 17)
Human pre-miR-519e is TCTCATGCAG TCATTCTCCA
AAAGGGAGCA CTTTCTGTTT GAAAGAAAAC AAAGTGCCTC
CTTTTAGAGT GTTACTGTTT GAGA.
(SEQ ID NO: 18)
Human pre-miR-489 GTGGCAGCTT GGTGGTCGTA
TGTGTGACGC CATTTACTTG AACCTTTAGG AGTGACATCA
CATATACGGC AGCTAAACTG CTAC.
(SEQ ID NO: 19)
Human pre-miR-124a is AGGCCTCTCT CTCCGTGTTC
ACAGCGGACC TTGATTTAAA TGTCCATACA ATTAAGGCAC
GCGGTGAATG CCAAGAATGG GGCTG.
(SEQ ID NO: 20)
Human pre-miR-218 is GTGATAATGT AGCGAGATTT
TCTGTTGTGC TTGATCTAAC CATGTGGTTG CGAGGTATGA
GTAAAACATG GTTCCGTCAA GCACCATGGA ACGTCACGCA
GCTTTCTACA.

### Sample Analysis

In certain embodiments, the disclosure relates to methods of analyzing samples for expression of microRNA or RNA disclosed herein. Typical methods are based on hybridization analysis of polynucleotides, and sequencing of polynucleotides. The most commonly used methods known in the art for the quantification of mRNA expression in a sample include northern blotting and in situ hybridization; RNAse protection assays; and reverse transcription polymerase chain reaction (RT-PCR). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). In certain embodiments, a microRNA detection agent such as a complementary nucleotide sequence can be labeled to allow detection in an imaging system, such as a positron emission tomography (PET) scan, Single-photon emission computed tomography (SPECT) or a similar type of scan by administering the labeled detection agent to the subject and then scanning the brain of the subject for binding. In those instances the detection agent may be labeled so as to only emit signal if bound to the microRNA.

Reverse Transcriptase PCR (RT-PCR) may be used to compare microRNA levels in different sample populations, in normal and tumor tissues, with or without drug treatment, to characterize patterns of gene expression, to discriminate between closely related microRNAs, and to analyze RNA structure. This method typically employs isolation of microRNA from a target sample, e.g., blood serum or brain fluid.

General methods for RNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. RNA may be isolated, for example, by cesium chloride density gradient centrifugation.

Typically, the methods entail the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

RT-PCR can be performed using commercially available equipment. In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data. 5′-Nuclease assay data are typically initially expressed as Ct, or the threshold cycle. As discussed above, fluorescence values are recorded during every cycle and represent the amount of product amplified to that point in the amplification reaction.

Real time quantitative PCR measures PCR product accumulation through a dual-labeled fluorigenic probe. Real time PCR is compatible both with quantitative competitive PCR, where internal competitor for each target sequence is used for normalization, and with quantitative comparative PCR using a normalization gene contained within the sample, or a housekeeping gene for RT-PCR.

Differential RNA expression can also be identified, or confirmed using the microarray technique. In this method, polynucleotide sequences of interest (microRNA) are plated, or arrayed, on a microchip substrate. The arrayed sequences are then hybridized with specific DNA probes from cells or tissues of interest. Just as in the RT-PCR method, the source of microRNA typically is total RNA

In a specific embodiment of the microarray technique, PCR amplified inserts of cDNA clones are applied to a substrate in a dense array. Preferably at least 10,000 nucleotide sequences are applied to the substrate. The microarrayed nucleic acids, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of nucleic acids. Microarray analysis can be performed by commercially available equipment, following manufacturer's protocols, such as by using the Affymetrix GenChip technology, or Incyte's microarray technology.

Serial analysis of gene expression (SAGE) is a method that allows the simultaneous and quantitative analysis of a large number of RNA, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

The MassARRAY (Sequenom, San Diego, Calif.) technology is an automated, high-throughput method of RNA expression analysis using mass spectrometry (MS) for detection. According to this method, following the isolation of RNA, reverse transcription and PCR amplification, the cDNAs are subjected to primer extension. The cDNA-derived primer extension products are purified, and dipensed on a chip array that is pre-loaded with the components needed for MALTI-TOF MS sample preparation. The various cDNAs present in the reaction are quantitated by analyzing the peak areas in the mass spectrum obtained.

Gene expression analysis by massively parallel signature sequencing (MPSS) as described in Brenner et al., Nature Biotechnology 18:630-634 (2000) provides a sequencing approach that combines non-gel-based signature sequencing with in vitro cloning of templates on separate microbeads. A microbead library of templates is constructed by in vitro cloning. This is followed by the assembly of a planar array of the template-containing microbeads in a flow cell at a high density. The free ends of the cloned templates on each microbead are analyzed simultaneously, using a fluorescence-based signature sequencing method that does not require fragment separation. This method has been shown to simultaneously and accurately provide, in a single operation, hundreds of thousands of sequences.

Immunohistochemistry methods are also suitable for detecting the expression levels of the microRNA markers. Thus, antibodies or antisera, preferably polyclonal antisera, and most preferably monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Typically methods of measurement of the microRNA include contacting a sample from a subject with a probe, which can be a nucleic acid-containing compound. Such nucleic acid-containing compound can be complementary to at least a portion, including at least 2, at least 3, at least 4, at least 5, at least 6, at least 7, at least 8, at least 9, at least 10, or at least 11 or more nucleic acids of the microRNA sequence. The probe can also be complementary to at least 50% m at least 60%, at least 70%, at least 80%, at least 90% or at least 95%, at least 98%, or more of the microRNA sequence. The probe can itself emit a signal or be linked to or bind to a compound that emits a signal, that can be measured, or can be used in a method of measurement such as during a PCR-based technique. In certain embodiments, the probe can be a compound that can be administered to a subject prior to measurement.

### Terms

The term “oligonucleotide” or “polynucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The polynucleotide may be generated in any manner, including chemical synthesis, DNA replication, reverse transcription, or a combination thereof.

The term “nucleic acid” refers to a polymer of nucleotides, or an oligonucleotide, as described above. The term is used to designate a single molecule, or a collection of molecules. Nucleic acids may be single stranded or double stranded, and may include coding regions and regions of various control elements.

The terms “complementary” and “complementarity” refer to oligonucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. The primer must be sufficiently long to prime the synthesis of extension products in the presence of the inducing agent. The exact lengths of the primers will depend on many factors, including temperature, source of primer and the use of the method.

The term “sequencing” refers to any number of methods may be used to identify the order of nucleotides a particular nucleic acid. The term “polymerase chain reaction” (“PCR”) refers to the method of K. B. Mullis U.S. Pat. Nos. 4,683,195, 4,683,202, and 4,965,188, that describe a method for increasing the concentration of a segment of a target sequence in a mixture. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

The term “reverse-transcriptase” or “RT-PCR” refers to a type of PCR where the starting material is mRNA. The starting mRNA is enzymatically converted to complementary DNA or “cDNA” using a reverse transcriptase enzyme. The cDNA is then used as a “template” for a “PCR” reaction.

### EXPERIMENTAL

### Example 1

### Altered Expression of Selective miRNAs in AD Brains

To explore the role of miRNAs in AD pathogenesis, miRNA expression profiles were investigated in brain tissues collected by the Emory ADRC Neuropathology Core. Using brain tissues of AD patients from the Emory ADRC Neuropathology Core, the expression of known miRNAs were analyzed and selective miRNAs were identified, including miR-572 and miR-650 that are altered specifically in AD (FIG. 1). Overall, 20 controls and 20 AD brain samples were analyzed, and found that miR-572 and miR-650 displayed statistically significant up-regulation in AD brains with 95% confidence intervals. To further determine whether the changes that we observed are AD-specific, the same number of frontotemporal dementia (FTD) samples were also analyzed and did not observe any significant difference between control and FTD samples. In addition, miR-9, a miRNA highly expressed in brain, did not change, indicating that the changes that we observed are specific.

### Example 2

### Identification of mRNA Targets of Selective miRNAs

Given that the expression levels of both miR-650 and miR-572 are elevated in AD brain tissues, studies to determine their mRNA targets were instigated, which might provide us insights on how these two pathophysiologically relevant miRNAs might contribute to AD pathogenesis. To identify the mRNA targets of miR-650 and miR-572, multiple bioinformatic software packages were utilized including TargetScan 5.0, miRanda and PicTar. For both miR-572 and miR-650, multiple mRNA targets were predicted by these programs.

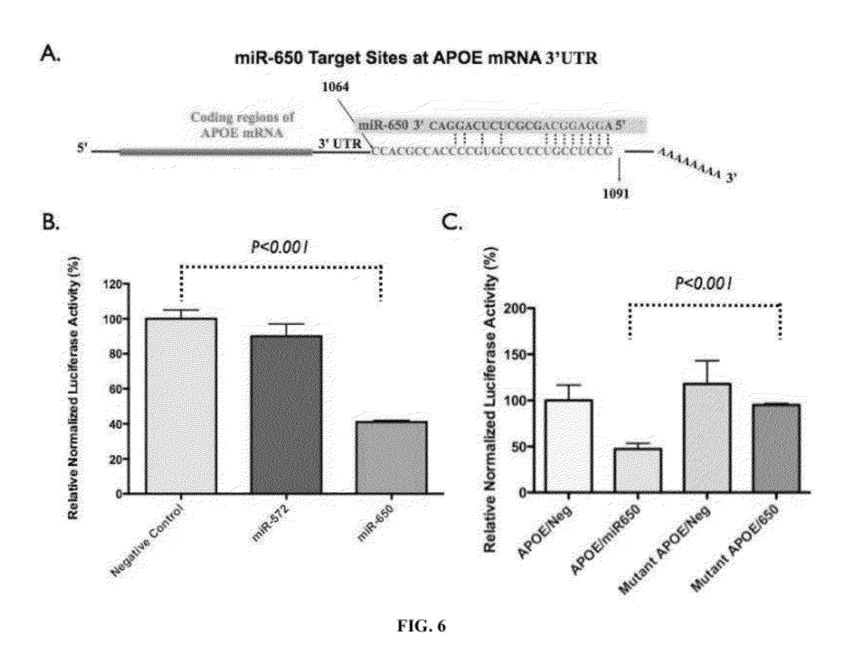

Both miR-572 and miR-650 were predicted to regulate the 3′-UTR of apolipoprotein E (APOE) mRNA. To test this possibility, a luciferase reporter construct with the 3′-UTR of APOE mRNA we generated. To determine whether either miR-572 or miR-650 could regulate the expression of APOE mRNA post-transcriptionally, we introduced the reporter constructs into HEK293 cells along with a siRNA-like miRNA duplex, which has been shown previously to function like endogenous miRNAs. Although both miR-572 and miR-650 were predicted to regulate APOE mRNA, only miR-650 could suppress the translation in our reporter system). Furthermore, to determine that the suppression we observed was directly due to miR-650, a mutant construct with the miR-650 target site deleted were generated. The deletion of miR-650 target site within the 3′-UTR of APOE abolished the suppression caused by miR-650, indicating that miR-650 could directly regulate the expression of APOE post-transcriptionally (FIG. 6C).

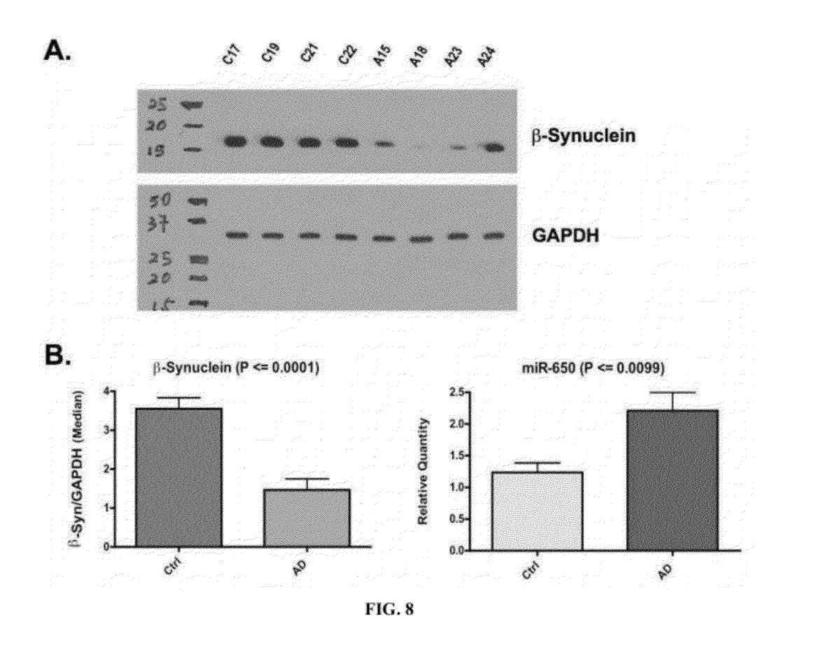

Interestingly the mRNA of β-synuclein was among the mRNAs that were predicted to be regulated by miR-650. To test whether miR-650 could regulate the expression of β-synuclein, a similar reporter construct for APOE was generated and tested it in HEK293 cells. MiR-650, but not miR-572, regulates the expression of the reporter gene with the 3′-UTR of β-synuclein (SCNB) (FIG. 8). Furthermore, this suppression is miR-650 specific since deletion of the miR-650 target site abolished the suppression. To determine whether the expression of miR-650 has any effect on the expression of endogenous β-synuclein mRNA, western blots were performed to determine the protein level of β-synuclein in both control and AD brain tissues. A significant reduction in the β-synuclein protein level was found and a significant increase in miR-650 levels in AD samples compared to controls. These results together suggest that miR-650 could regulate the expression of β-synuclein post-transcriptionally.

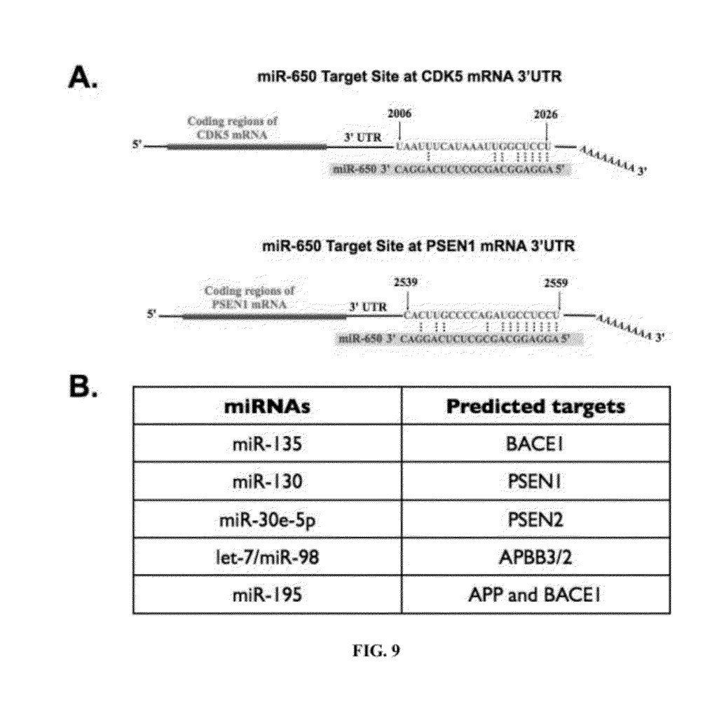

Both presenilin 1 (PSEN1) and CDK5, were predicted by bioinformatics to be regulated by miR-650. The prediction by our bioinformatic analyses suggest that miR-650 could potentially regulate all of these genes implicated in AD pathogenesis raises the possibility that miR-650 could function as a master miRNA to fine-tune the expression of multiple genes implicated in AD pathogenesis. miRNA profiling studies have also revealed that miR-135, miR-130, miR-30e-5p, let-7/miR-98 and miR-195 displayed altered expression in AD brain tissues. Bioinformatic analyses have identified several genes that have been implicated in AD pathogenesis as well (FIG. 9).

1. A method of screening a subject for Alzheimer's Disease comprising
a) measuring the quantity of at least one microRNA in a brain of a subject by contacting a sample of tissue from the subject with a microRNA measuring probe;
b) comparing the measured amount of microRNA to a predetermined amount of microRNA indicative of Alzheimer's Disease; and
c) if the measured amount of microRNA is increased as compared to the predetermined amount, categorizing the sample as indicative of Alzheimer's Disease in the subject.

2. The method of claim 1 further comprising administering the probe to the subject.

3. The method of claim 1 wherein a sample of tissue is taken from the subject.

4. The method of claim 1 wherein the probe comprises a nucleic acid complementary to at least a portion of the microRNA.

5. The method of claim 1, further comprising the step of recording whether the sample is indicative of Alzheimer's Disease in the subject.

6. The method of claim 1, wherein the microRNA is one or more selected from the group miR-515-3p, miR-21, miR-576, miR-490, miR-187, miR-449, miR-646, miR-409-5p, miR-518e, miR-517c, miR-320, miR-564, miR-191, miR-142-5p, miR-501, miR-519e, miR-489, miR-124a, miR-218, and miR-650.

7. The method of claim 1, wherein the quantity of at least two microRNA is measured.

8. The method of claim 1, wherein the quantity of at least three microRNA is measured.

9. The method of claim 1, wherein the sample is collected from a subject suspected of or at risk of having Alzheimer's disease.

10. The method of claim 1, wherein the sample is a blood sample obtained from pulmonary or systemic circulation.


SEQUENCE LISTING (20 sequences)

SEQ ID NO: 1 - DNA, 96 bp, Homo sapiens
cagtgctggggtctcaggaggcagcgctctcaggacgtcaccaccatggcctgggctctgctcctcctcaccctcctcactcagggcacaggtgat

SEQ ID NO: 2 - DNA, 83 bp, Homo sapiens
tctcatgcagtcattctccaaaagaaagcactttctgttgtctgaaagcagagtgccttcttttggagcgttactgtttgaga

SEQ ID NO: 3 - DNA, 72 bp, Homo sapiens
tgtcgggtagcttatcagactgatgttgactgttgaatctcatggcaacaccagtcgatgggctgtctgaca

SEQ ID NO: 4 - DNA, 98 bp, Homo sapiens
tacaatccaacgaggattctaatttctccacgtctttggtaataaggtttggcaaagatgtggaaaaattggaatcctcattcgattggttataacca

SEQ ID NO: 5 - DNA, 128 bp, Homo sapiens
tggaggccttgctggtttggaaagttcattgttcgacaccatggatctccaggtgggtcaagtttagagatgcaccaacctggaggactccatgctgttgagctgttcacaagcagcggacacttcca

SEQ ID NO: 6 - DNA, 109 bp, Homo sapiens
ggtcgggctcaccatgacacagtgtgagacctcgggctacaacacaggacccgggcgctgctctgacccctcgtgtcttgtgttgcagccggagggacgcaggtccgca

SEQ ID NO: 7 - DNA, 91 bp, Homo sapiens
ctgtgtgtgatgagctggcagtgtattgttagctggttgaatatgtgaatggcatcggctaacatgcaactgctgtcttattgcatataca

SEQ ID NO: 8 - DNA, 94 bp, Homo sapiens
gatcaggagtctgccagtggagtcagcacacctgcttttcacctgtgatcccaggagaggaagcagctgcctctgaggcctcaggctcagtggc

SEQ ID NO: 9 - DNA, 79 bp, Homo sapiens
tggtactcggggagaggttacccgagcaactttgcatctggacgacgaatgttgctcggtgaaccccttttcggtatca

SEQ ID NO: 10 - DNA, 88 bp, Homo sapiens
tctcaggctgtgaccctctagagggaagcgctttctgttggctaaaagaaaagaaagcgcttcccttcagagtgttaacgctttgaga

SEQ ID NO: 11 - DNA, 95 bp, Homo sapiens
gaagatctcaggcagtgaccctctagatggaagcactgtctgttgtctaagaaaagatcgtgcatccttttagagtgttactgtttgagaaaatc

SEQ ID NO: 12 - DNA, 82 bp, Homo sapiens
gcctcgccgccctccgccttctcttcccggttcttcccggagtcgggaaaagctgggttgagagggcgaaaaaggatgtggg

SEQ ID NO: 13 - DNA, 94 bp, Homo sapiens
cgggcagcgggtgccaggcacggtgtcagcaggcaacatggccgagaggccggggcctccgggcggcgccgtgtccgcgaccgcgtaccctgac

SEQ ID NO: 14 - DNA, 92 bp, Homo sapiens
cggctggacagcgggcaacggaatcccaaaagcagctgttgtctccagagcattccagctgcgcttggatttcgtcccctgctctcctgcct

SEQ ID NO: 15 - DNA, 87 bp, Homo sapiens
gacagtgcagtcacccataaagtagaaagcactactaacagcactggagggtgtagtgtttcctactttatggatgagtgtactgtg

SEQ ID NO: 16 - DNA, 84 bp, Homo sapiens
gctcttcctctctaatcctttgtccctgggtgagagtgctttctgaatgcaatgcacccgggcaaggattctgagagggtgagc

SEQ ID NO: 17 - DNA, 84 bp, Homo sapiens
tctcatgcagtcattctccaaaagggagcactttctgtttgaaagaaaacaaagtgcctccttttagagtgttactgtttgaga

SEQ ID NO: 18 - DNA, 84 bp, Homo sapiens
gtggcagcttggtggtcgtatgtgtgacgccatttacttgaacctttaggagtgacatcacatatacggcagctaaactgctac

SEQ ID NO: 19 - DNA, 85 bp, Homo sapiens
aggcctctctctccgtgttcacagcggaccttgatttaaatgtccatacaattaaggcacgcggtgaatgccaagaatggggctg

SEQ ID NO: 20 - DNA, 110 bp, Homo sapiens
gtgataatgtagcgagattttctgttgtgcttgatctaaccatgtggttgcgaggtatgagtaaaacatggttccgtcaagcaccatggaacgtcacgcagctttctaca